(19) United States
(12) Patent Application Publication
(10) Pub. No.: US 2014/0363827 A1
(43) Pub. Date: Dec. 11, 2014

(54) APPARATUS FOR IDENTIFYING CHARACTERISTIC OF LIQUID AND THE METHOD THEREOF

(71) Applicant: National Cheng Kung University, Tainan City (TW)

(72) Inventors: Shu-Hsien Liao, Taichung City (TW); Chuan-Fa Chang, Tainan City (TW); Hsien Chang Chang, Tainan City (TW)

(21) Appl. No.: 14/297,143

(22) Filed: Jun. 5, 2014

(30) Foreign Application Priority Data
Jun. 6, 2013 (TW) 102120203

(51) Int. Cl. G01N 33/80

(52) U.S. Cl. CPC G01N 33/80
USPC 435/7.25; 435/287.2; 435/372

(57) ABSTRACT

An apparatus and a method for identifying the characteristics of a liquid sample due to capillary force are disclosed. The apparatus and the method spread the blood sample (which is obtained from the blood of a subject, or a mixture containing the blood of two different subjects) having an agglutination portion in a distribution space due to the capillary force.

### CROSS-REFERENCE TO RELATED APPLICATION AND CLAIM OF PRIORITY

The application claims the benefit of Taiwan Patent Application No. 102120203, filed on Jun. 6, 2013, at the Taiwan Intellectual Property Office, the disclosures of which are incorporated herein in their entirety by reference.

### TECHNICAL FIELD

The present disclosure is directed to an apparatus and a method for identifying characteristics of a liquid sample (e.g. blood sample) using capillary force. The apparatus and the method spread the blood sample (which is obtained from a subject, or two different subjects) having an agglutination portion in a distribution space (e.g. a channel) due to the capillary force.

### BACKGROUND

Blood banks at hospitals store and test blood, and preserve it when needed. In addition to the basic ABO blood typing, another important work is crossmatching (of bloods). The crossmatching is very important because that is the pre-process before a blood transfusion. A medical technologist has to complete the crossmatching of a donor's and a recipient's blood cells and plasma before the recipient receives the donor's blood. Presently, the tests for blood typing and crossmatching are manually performed in the hospital, which requires substantial time, work, and cost. In addition, if unmatched blood is found, the recipient's blood in the unmatched blood must be further tested to find out what kind of irregular antibody exists therein to provide suitable blood to the recipient. If the patient needs an urgent blood transfusion, the medical technologist will get a new blood bag to reconfirm the crossmatching.

Regular antibody tests for blood include the identification of ABO and Rh blood types. ABO blood types were discovered by Dr. Karl Landsteiner in 1900, they were the first blood type system found, and they are the most important ones. The identification of ABO blood types is performed by means of the antigen located on the cell membrane of a red blood cell (RBC). Specifically, a human with the A blood type has A antigen on RBC and anti-B antibody in plasma or serum, a human with the B blood type has B antigen on RBC and anti-A antibody in plasma or serum, a human with the AB blood type has A antigen and B antigen on RBC and has neither anti-A antibody or anti-B antibody in plasma or serum, and a human with the O blood type does not have any A antigen and B antigen on RBC but has anti-A antibody and anti-B antibody in plasma or serum. Therefore, a human who has the O blood type can transfuse his red blood cells to anyone. However, if a human with the A blood type transfuses his blood to one with the B blood type, the anti-B antibody in the plasma of the donor will cause the recipient's red blood cells to agglutinate, and may even cause the recipient to die. Accordingly, the identification of blood types is critically important.

In the blood bank at the hospital, the identification of ABO blood types must include a cell grouping and a serum grouping. The cell grouping refers to using the anti-A antibody and anti-B antibody to identify the ABO blood types. For example, an agglutination of type A blood would be caused by an anti-A antibody but not an anti-B antibody, and this blood type can therefore be identified. The serum grouping refers to using A cells of A blood type and B cells of B blood type (which are usually disposed on RBCs) to identify the antibody species in the plasma or serum to determine the ABO blood types. For example, an agglutination in plasma or serum of type B blood would be caused by the A antigen disposed on RBC but not the B antigen, and this blood type can therefore be identified. The results of cell grouping and serum grouping must be consistent or else the blood type cannot be determined.

Another regular blood type system is the Rh blood type system, which is extremely complex because it includes 46 antigens. The expression of these antigens is determined by two genes, rhd and rhce, located on chromosome 1p36.11. The unglycosylated proteins, RHD and RHCE respectively expressed from rhd and rhce, only exist on the surface of RBC, form D antigen, and can therefore be identified. The Rh+ blood type denotes that there is D antigen on the surface of RBC and there is no anti-D antibody in the plasma or serum. The Rh− blood type denotes that there is no D antigen on the surface of RBC, and anti-D antibody may exist in the plasma or serum (one out of 3,000 people has the anti-D antibody in their plasma or serum). Therefore, Rh+/− blood types can be identified using the anti-D antibody.

Crossmatching is an important step before blood transfusions in the hospital, and includes major and minor crossmatching tests. The major crossmatching test refers to mixing the plasma or serum of a recipient (patient) and the blood cells of a donor to determine whether there is an irregular antibody in the plasma or serum of the recipient. The minor crossmatching test refers to mixing the blood cells of a recipient and the plasma or serum of a donor to determine whether there is an abnormal antigen on the RBCs of the recipient. The major and minor crossmatching tests determine whether the donor's blood is suitable to transfuse to the recipient. If the results of the major and minor crossmatching tests are incorrect, a chronic or acute transfusion reaction may occur in the recipient.

In developing countries, not every basic test mentioned above is performed before a blood transfusion in the hospital, and therefore a safe procedure for blood transfusion is unachievable. In addition, in some emergency situations, such as on a battle field or at the scene of a disaster, the rapid procedures and correct results for the above-mentioned basic tests are necessary for the person who needs an emergency blood transfusion, otherwise an acute or chronic transfusion reaction, e.g. hematuria, liver failure, and kidney failure, may occur after unmatched blood is transfused to that person. Moreover, an irregular antibody may cause transfusion related acute lung injury (TRALI) which may lead to the death of the recipient/patient.

After extensive experiments and persistent research, the applicant has finally conceived an apparatus for identifying characteristics of liquids and the method thereof.

### SUMMARY

The present disclosure is directed to an apparatus and a method for identifying characteristics of a liquid sample (e.g. blood sample) using capillary force. The apparatus and the method spread the blood sample (which is obtained from the blood of a subject, or the mixture containing the blood of two different subjects) having an agglutination portion in a distribution space (e.g. a channel) using the capillary force.

In another aspect, the present disclosure discloses a test chip, comprising: a flow channel having an inlet, an inlet surface and a channel surface, wherein at least one of the inlet surface and the channel surface has an antibody disposed thereon, the inlet receives a blood sample having an antigen, and the blood sample flows into the channel due to a capillary force so that the antibody binds to the antigen and flows along with the blood sample.

In another aspect, the present disclosure discloses a method for identifying a blood type, comprising steps of: providing a first and a second blood samples both obtained from a human and respectively having a first and a second red blood cells; providing a first and a second distribution spaces; providing an anti-A antibody and an anti-B antibody; mixing the first and the second blood samples with the anti-A antibody and the anti-B antibody respectively; causing the first blood sample mixed with the anti-A antibody to be distributed in the first distribution space due to a first capillary force; and causing the second blood sample mixed with the anti-B antibody to be distributed in the second distribution space due to a second capillary force.

In another aspect, the present disclosure discloses a method for identifying a blood type, comprising steps of: providing a first and a second blood samples both obtained from a human and respectively having a first and a second serum antibodies; providing a first and a second distribution spaces; providing an A antigen and a B antigen; mixing the first and the second blood samples with the A antigen and the B antigen respectively; causing the first blood sample mixed with the A antigen to be distributed in the first distribution space due to a first capillary force; and causing the second blood sample mixed with the B antigen to be distributed in the second distribution space due to a second capillary force.

In another aspect, the present disclosure discloses a method for identifying a blood type, comprising steps of: providing a first and a second blood samples both obtained from a human and respectively having a first and a second serum antibodies; providing a first and a second distribution spaces; providing an A antigen and a B antigen; mixing the first and the second blood samples with the A antigen and the B antigen respectively; causing the first blood sample mixed with the A antigen to be distributed in the first distribution space due to a first capillary force; and causing the second blood sample mixed with the B antigen to be distributed in the second distribution space due to a second capillary force.

On another aspect, the present disclosure discloses a crossmatching method, comprising steps of: providing a first blood sample of a first human, wherein the first sample includes one of a plasma portion and a serum portion; providing a second blood sample of a second human being different from the first human, wherein the second sample includes a blood cell portion; mixing the first and the second blood samples to form a mixed blood sample; providing a distribution space; and causing the mixed blood sample to be distributed in the distribution space using a capillary force.

### BRIEF DESCRIPTION OF THE DRAWINGS

FIGS. 1A, 1B, and 1C are diagrams showing an embodiment of the present apparatus.

FIGS. 2, 3, 4, and 5 are diagrams showing embodiments demonstrating the results of the distribution of blood samples in the channels.

### DETAILED DESCRIPTION

The present disclosure can be fully understood and accomplished by the skilled person according to the following embodiments. However, the practice of the present method is not limited to the following embodiments.

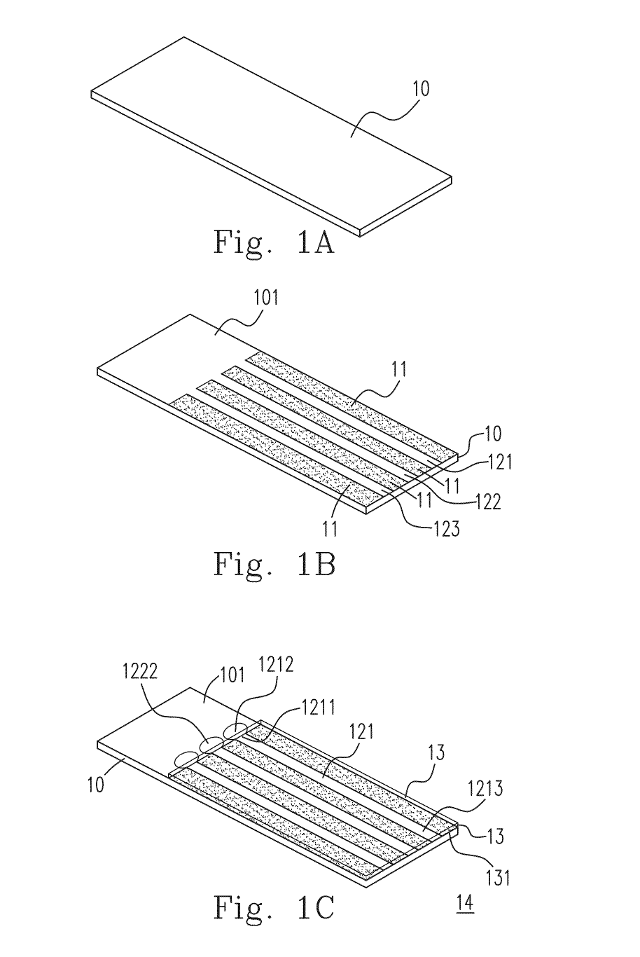

Please refer to FIGS. 1A, 1B, and 1C which show an embodiment of the present apparatus. FIG. 1A shows a substrate 10 of the present apparatus. As shown in FIG. 1B, plural middle layers 11 are configured on substrate 10 to define thereon a first, a second and a third flow channels 121, 122 and 123. As shown in FIG. 1C, by covering a upper layer on middle layers 11, a chip 14, the embodiment of the present apparatus is completed in which the first, second and third flow channels 121, 122 and 123 are configured.

In some embodiments, substrate 10 is made of glass, or acrylic or plastic material, and a surface treatment can further be performed on these substrates to increase their hydrophilicity. In addition, conductive material can be coated onto these substrates to raise their conductivity, and an appropriate electric power can therefore be applied thereto to control the temperature of these substrates. Middle layers 11 can be made using double-sided adhesive with suitable thickness to rapidly bond substrate 10 and upper layer 13 (cover layer). Middle layers 11 can also be made using another adhesive (e.g. optical glue) or material (such as plastic or photoresist) that bonds substrate 10 to upper layer 13. In order to make the observation of the sample flowing/distributing in the channels easier, upper layer 13 is usually a transparent plan and can further have graduations thereon and/or a magnifier configured therein.

In some embodiments, substrate 10 and the first, second and third flow channels 121, 122 and 123 are form as one piece, and therefore substrate 10 and upper layer 13 can be bonded by simply method, such as adhesive, hot-melt and engagement, to complete the configuration of the channels.

Each of the first, second and third flow channels 121, 122 and 123 has an inlet and a far end relative to the inlet. For example, the first flow channel 121 has an inlet 1211, an inlet surface 1212 and a far end 1213. In an embodiment, at least one of a surface of channel 121 and inlet surface 1212 has an antibody temporarily bonded thereon. The temporarily bonded antibody means that the antibody is temporarily bonded on the surface, and if there is fluid (sample) driven by a force, e.g. a capillary force, to flow thereon, the antibody leave the surface and move along the direction of the flowing fluid. In an embodiment, the temporarily bonded antibody is configured by dropping a liquid containing the antibody on the inlet/channel surface and then allowing the liquid to dry.

In an embodiment, substrate 10 and upper layer 13 have an upper surface and a lower surface. The inlet surfaces and the channel surfaces are located on the upper surface 101 of substrate 10. In addition, both the upper surface 101 of substrate 10 and the lower surface 131 of upper layer 13 are continuous, extending and impermeable planes, and the channels 121, 122 and 123 are formed therebetween.

In another embodiment, the antibody is temporarily bonded on the lower surface 131 of upper layer 13. Therefore, the lower surface 131 can be seen the channel surface on which the antibody is disposed.

In an embodiment, when chip 14 is applied to the identification of characteristics of a blood sample, if there is an antigen, which can specifically bind with the antibody temporarily bonded on the inlet/channel surface, in the blood sample, the antigen will specifically bind with the antibody. Specifically, if the antigen is the one that exists on RBC for the determination of blood type (e.g. A or B antigen), chip 14 is then applicable to the identification of the blood type.

In detail, if the blood sample is type A whole blood, this blood sample contains anti-B antibody and A antigen located on the surface of RBC. When this blood sample is disposed at inlet 1211 and the surface of channel 121 and/or inlet surface 1212 having anti-A antibody temporarily bonded thereon, this blood sample will be drawn into channel 121 because of the capillary force generated in channel 121 and then flow to the far end 1213. Due to the capillary force, the RBCs having the A antigen in the blood sample will flow along channel 121 from inlet 1211 to the far end 1213. The flowing blood sample/RBCs cause the A antigen located on the RBC to collide and mix with the anti-A antibody, which therefore generates a blood cell agglutination reaction. Because the anti-A antibodies are temporarily bonded on the surface of channel 121 and/or inlet surface 1212, they will be continuously specifically bound with the A antigen on the RBC and make the RBCs agglutination. Those agglutinated RBCs will be carried to the far end 1213 by the flowing blood sample and will be further condensed and revealed at the far end 1213 (at the area of the end opposite to and far from the end of channel 121 which is the inlet 1211's location). The agglutinated RBCs may also be carried by the flowing blood sample and revealed at a specific location (e.g. at the front, front-middle, middle, middle-back or back part) of the channel being at a distance from the inlet, depending on, for example, flow rate or viscosity of the flowing blood sample and agglutination degree of the agglutination RBCs.

Based on the above, in the case of a type B blood sample, containing anti-A antibody and RBC having B antigen, no agglutination will occur in the blood sample in channel 121. That is, the type B blood sample will be uniformly distributed in channel 121.

In an embodiment, anti-A antibody is temporarily bonded on the surface of channel 121 and/or inlet surface 1212, anti-B antibody is temporarily bonded on the surface of channel 122 and/or inlet surface 1222, and this chip is applicable for the identification of ABO blood types. Specifically, if a tested blood sample is drawn into and flows in both channels 121 and 122, and: is only agglutinated in channel 121, this tested blood sample is type A blood; is only agglutinated in channel 122, this tested blood sample is type B blood; is agglutinated in both of channels 121 and 122, this tested blood sample is type AB blood (which is because the RBC of type AB blood has both the A and the B antigens); and is agglutinated in neither channel 121 nor 122, this tested blood sample is type O blood (which is because the RBC of type 0 blood has neither the A nor the B antigen).

Rh+ type blood contains RBC having D antigen thereon, and the RBC of Rh− type blood has no D antigen thereon. Accordingly, in an embodiment, anti-A antibody is temporarily bonded on the surface of channel 121 and/or inlet surface 1212, anti-B antibody is temporarily bonded on the surface of channel 122 and/or inlet surface 1222, anti-D antibody is temporarily bonded on the surface of channel 123 and/or inlet surface 1223, and this chip can be used to rapidly and simultaneously identify both ABO and Rh blood types.

In an embodiment, more than two species of antibodies are temporarily bonded on the surface of a single channel and/or its inlet surface. For example, both of anti-A and anti-B antibodies are temporarily bonded on the surface of the single channel and/or its inlet surface, and this kind of chip can be used to rapidly identify whether a blood sample is type O blood (where there is not any agglutination in the single channel if the blood sample is type O blood).

In an embodiment, blood samples obtained from a human are mixed with different species of antibodies (e.g. anti-A, anti-B and anti-D antibodies) and these mixed blood samples flow in separate channels, such as those disposed on chip 14. Through the capillary forces generated in the separate channels, the mixed blood samples are distributed in the separate channels and it can therefore be observed whether there is any agglutination in each separate channel to determine the ABO and/or Rh blood types.

In an embodiment, RBCs having A antigen or B antigen (respectively substitutes for anti-A and anti-B antibodies) are temporarily bonded on the inlet surface and/or channel surface, or mixed with a blood, plasma or serum sample. Then, the mixed sample (containing the RBCs and blood, plasma or serum sample) flows and is distributed in the channel because of the capillary force generated by the channel. Therefore if the mixed sample has agglutination, the agglutination will appear and be separated in the channel. Such serum typing can used to identify blood type, e.g. ABO blood type. In regard to the blood sample identified by serum typing, if the blood sample is whole blood or plasma, the anti-A or anti-B antibody contained therein is called plasma antibody, and if the blood sample is serum, the anti-A or anti-B antibody contained therein is called serum antibody.

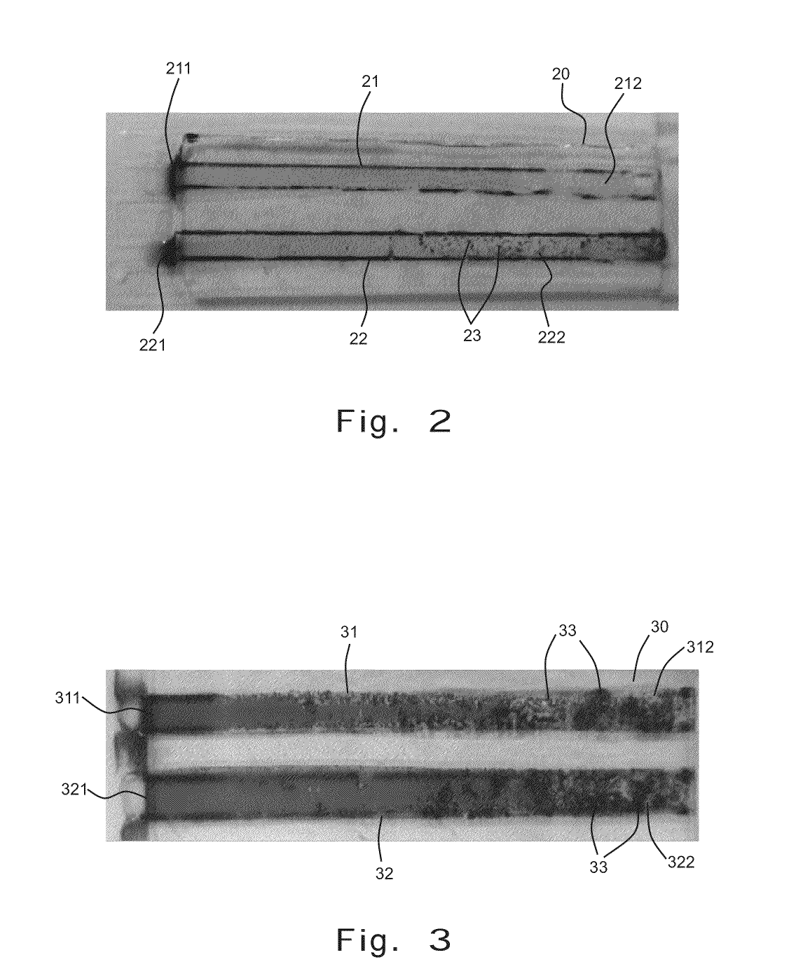

Please refer to FIG. 2 which is a diagram showing the results of identifying blood types. In FIG. 2, anti-A and anti-B antibodies were temporarily bonded on the surfaces of flow channels 21 and 22 respectively. Each of flow channels 21 and 22, having a size of 5 cm×2 mm×30 μm and configured on chip 20, draws a type A blood sample (10 μL, washed and diluted to have a hematocrit of 3-5%). Therefore, after the type A blood sample flows from inlets 211 and 221 to the far ends 212 and 222, the agglutination of RBCs occurs and can be observed (i.e. agglutination portion 23) at the far end 222 of channel 22 which has anti-A antibody therein. At the same time, there is no agglutination of RBCs observed in channel 21.

Please refer to FIG. 3 which is a diagram showing the results of identifying blood types. In FIG. 3, anti-A and anti-B antibodies were temporarily bonded on the surfaces of flow channels 31 and 32 respectively. Each of flow channels 31 and 32, having a size of 5 cm×2 mm×30 μm and configured on chip 30, draws a type AB blood sample (10 μL, whole blood). After the type AB blood sample flows from inlets 311 and 321 to the far ends 312 and 322, the agglutinations of RBCs occur and can be observed (i.e. agglutination portion 33) at both of far ends 312 and 322 of channels 31 and 32 which have anti-A and anti-B antibodies therein respectively.

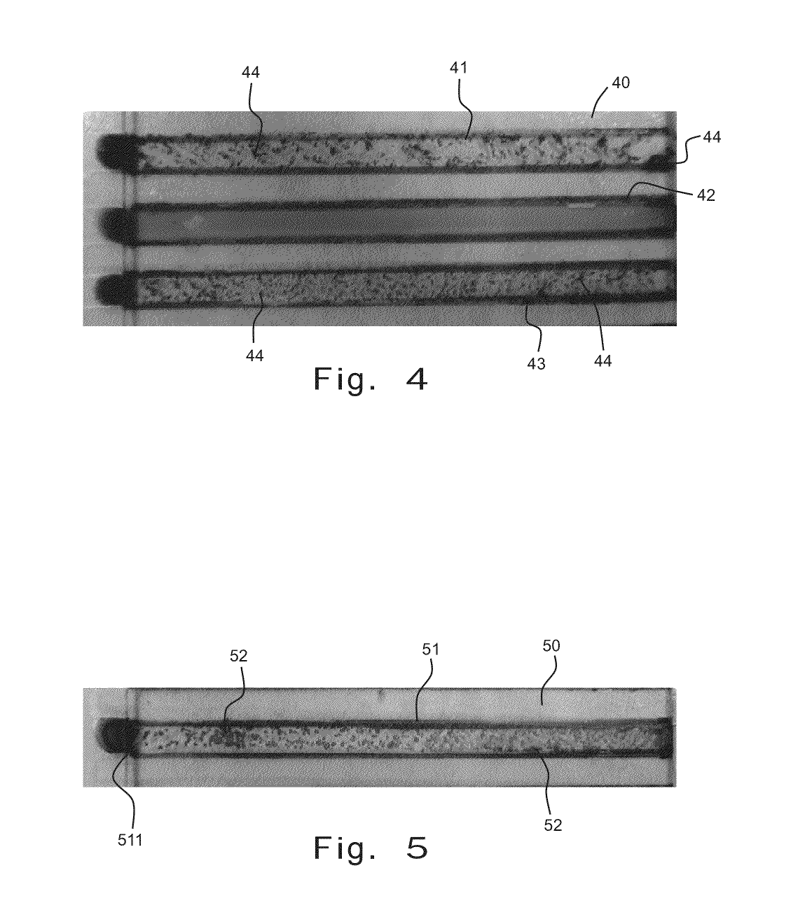

Please refer to FIG. 4 which is a diagram showing the results of identifying blood types. In FIG. 4, each of three type A and Rh+ blood samples are mixed with anti-A, anti-B and anti-D antibodies before the mixed samples were injected into the channel, where the blood sample mixed with anti-A and anti-D antibodies will have agglutinations of RBCs therein. These mixed blood samples are drawn into flow channels 41, 42, and 43, and are distributed therein by capillary forces, where each flow channel 41, 42, and 43 has a size of 5 cm×2 mm×30 μm and is configured on chip 40. Because the blood samples mixed with anti-A and anti-D antibodies have agglutinations of RBCs before they were drawn into the channel, agglutinations of RBCs 44 are spread and distributed in the entire channels 41 and 43. Meanwhile, there is no agglutination of RBCs observed in channel 42 (in which the blood sample mixed with anti-B antibody flowed and was distributed).

Please refer to FIG. 5 which is a diagram showing the result of crossmatching. In FIG. 5, a mixed blood sample, obtained by uniformly mixing an RBC suspension of type B blood (10 μL, obtained from a first human) and plasma of type A blood (20 μL, obtained from a second human different from the first human), is drawn into and distributed in channel 51 having a size of 5 cm×2 mm×30 μm and configured on chip 50. Because there are agglutinations of RBCs, caused by the RBCs of the first human having the B antigen thereon and the anti-B antibody in the plasma of the second human, in the mixed blood sample, after the mixed blood sample is drawn into channel 51 from inlet 511 and flows and is distributed in channel 51, the agglutinations of RBCs (agglutination portion 52) are spread and distributed in the entire channel 52. Based on the result of crossmatching shown in FIG. 5, it can be seen that the blood of the first and the second human do not match.

In an embodiment, a crossmatching method is disclosed. The crossmatching method comprises steps of providing a first blood sample of a first human, wherein the first sample includes one of a plasma portion and a serum portion, providing a second blood sample of a second human being different from the first human, wherein the second sample includes a blood cell portion, mixing the first and the second blood samples to form a mixed blood sample, providing a distribution space, and causing the mixed blood sample to be distributed in the distribution space because of capillary force. In the crossmatching method, both the plasma portion and the serum portion have antibodies, the blood cell portion has RBCs, and the RBCs have antigens thereon. If the antibodies and antigens are specifically bound in the mixing step, an agglutination reaction would therefore occur. The agglutination reaction makes the RBCs agglutinate, and the agglutinated RBCs form an agglutination portion in the mixed blood sample. In addition, the crossmatching method comprises a step of distributing the mixed blood sample via the distribution space by the capillary force to further separate the agglutination portion into several small and isolated agglutination portions. The small agglutination portions can easily be identified and observed. Further, because there is an agglutination portion in the mixed blood sample, it can be seen the blood of the first human and the second human do not match.

Alternatively, if the antibodies and antigens fail to specifically bind in the mixing step, the agglutination reaction will not occur. In this situation, after the mixed blood sample is distributed in the distribution space, no agglutination portion will be identified/observed. That is, the blood of the first human and the second human match.

In an embodiment, the test chip has one or more isolated mix tank(s) which do not communicate with the flow channel. The mix tank has a surface which is modified with temporarily bonded antibodies or antigens.

In an embodiment, a tank is configured on the test chip and at the inlet of the channel. The tank communicates with the flow channel, and a surface of the tank can be seen as the inlet surface. The chip of this embodiment is not only convenient for mixing the blood sample and the antibodies or antigens modified on the inlet surface, but also avoids the blood sample flowing to another flow channel, which would cause contamination.

In an embodiment, an inlet can communicate with plural flow channels, and the surfaces of the flow channels are modified with different antibodies and/or antigens. For example, the inlet that communicates with the plural flow channels has a first channel and a second channel, and the surface of the first channel and the surface of the second channel are respectively modified with anti-A antibodies and anti-B antibodies. The test chip having this configuration can simultaneously identify various characteristics of a single blood sample. In addition, the plural flow channels can have a forking shape, a symmetrical shape or a radial shape.

In an embodiment, the height of the flow channel (between the upper surface of substrate and the lower surface of upper layer) is less than 1 mm, preferably is less than 100 μm, more preferably is less than 50 μm, and more preferably is less than 30 μm. In addition, the capillary force generated by the flow channel can be increased by extending the length of the flow channel. Furthermore, a single flow channel can be linear, curved or a combination thereof.

In an embodiment, irregular antibodies, such as the antibody against C, E, c, e, M, N, S, s, P₁, Le^{a}, Le^{b}, K, k, Fy^{a}, Fy^{b}, Jk^{a}, Jk^{b}, Mi^{a}, Di^{a} antigen, can be used to identify the corresponding antigen in the blood sample.

In an embodiment, a sample is mixed with sample blood, and distributed in the distribution space to identify whether it would cause any agglutination in the sample blood. For example, the sample could be a medical substance.

In an embodiment, plural distribution spaces (e.g. channels) are configured on a single chip, where the surfaces/inlet surfaces of the plural distribution spaces are modified by appropriate temporarily bounded antigen, antibody and/or specific binding material. In addition, indicators are marked at appropriate locations (e.g. near the distribution spaces) on the chip that show what kind of material is modified on the respective surfaces/inlet surfaces, and/or what kind of property of blood (e.g. ABO blood type) can be identified by the distribution spaces. Such a chip can therefore be a customized one for mass production for blood identification.

The present disclosure also discloses an embodiment to identify whether there is an agglutination part in a liquid sample. Specifically, in the embodiment, the sample flows and is distributed in a liquid distribution space because of capillary force. Therefore, if the agglutination part exists in the sample, it will be detected and observed in the distribution space.

### Embodiments

Embodiment 1 is a test chip, comprising: a flow channel having an inlet, an inlet surface and a channel surface, wherein at least one of the inlet surface and the channel surface has an antibody disposed thereon, the inlet receives a blood sample having an antigen, and the blood sample flows in the channel via a capillary force so that the antibody binds to the antigen and flows along with the blood sample.

Embodiment 2 is a test chip according to Embodiment 1, wherein the blood sample enters the flow channel from the inlet, and the antibody is mixed with the blood sample and specifically bound with the antigen.

Embodiment 3 is a test chip according to Embodiments 1 or 2, wherein the flow channel further has a far end opposite to the inlet, and the antibody bound with the antigen flows along with the blood sample to the far end.

Embodiment 4 is a test chip according to any of Embodiments 1 to 3, and further comprises a substrate and an upper cover, and the channel is configured between the substrate and the upper cover.

Embodiment 5 is a test chip according to Embodiment 4, wherein the substrate has an upper surface, the upper cover has a lower surface, the upper surface and the lower surface are continuous planes, and the channel is configured between the upper surface and the lower surface.

Embodiment 6 is a test chip according to Embodiment 5, wherein the flow channel has a channel height being a distance between the upper surface and the lower surface and less than 1 mm.

Embodiment 7 is a test chip according to Embodiment 4, wherein the substrate has an upper surface, the upper cover has a lower surface, and the inlet surface and the channel surface are located on the upper surface.

Embodiment 8 is a test chip according to Embodiment 4, wherein the substrate has an upper surface, the upper cover has a lower surface, the inlet surface is located on the upper surface, and the channel surface is located on the lower surface.

Embodiment 9 is a test chip according to any of Embodiments 1 to 8, wherein the blood sample has a red blood cell having the antigen thereon, and the antibody is bound with the antigen so as to form an agglutination.

Embodiment 10 is a method for identifying a blood type, comprising steps of: providing a first and a second blood samples both obtained from a human and respectively having a first and a second red blood cells; providing a first and a second distribution spaces; providing an anti-A antibody and an anti-B antibody; mixing the first and the second blood samples with the anti-A antibody and the anti-B antibody respectively; causing the first blood sample mixed with the anti-A antibody to be distributed in the first distribution space due to a first capillary force; and causing the second blood sample mixed with the anti-B antibody to be distribute in the second distribution space due to a second capillary force.

Embodiment 11 is a method according to Embodiment 10, wherein the first and the second distribution spaces are separately disposed without being connected to each other.

Embodiment 12 is a method according to Embodiments 10 or 11, and further comprises steps of providing a third blood sample obtained from the human and having a third red blood cell; providing a third distribution space independently disposed without being connected to either the first distribution space or the second distribution space; providing an anti-D antibody; mixing the third blood sample with the anti-D antibody; and causing the third blood sample mixed with the anti-D antibody to be distributed in the third distribution space due to a third capillary force.

Embodiment 13 is a method for identifying a blood type, comprising steps of: providing a first and a second blood samples both obtained from a human and respectively having a first and a second serum antibodies; providing a first and a second distribution spaces; providing an A antigen and a B antigen; mixing the first and the second blood samples with the A antigen and the B antigen respectively; causing the first blood sample mixed with the A antigen to be distributed in the first distribution space due to a first capillary force; and causing the second blood sample mixed with the B antigen to be distributed in the second distribution space due to a second capillary force.

Embodiment 14 is a method according to Embodiment 13, wherein the first and the second distribution spaces are separately disposed without being connected to each other.

Embodiment 15 is a method according to Embodiments 13 or 14, wherein each of the A and the B antigens is disposed on a surface of a red blood cell.

Embodiment 16 is a method for detecting an agglutination portion in a liquid, comprising steps of: providing a distribution space; providing the liquid; and causing the liquid to be distributed in the distribution space using a capillary force to reveal the agglutination portion.

Embodiment 17 is a method according to Embodiment 16, wherein the liquid is a mixture mixing therein plural red blood cells of a first human and one of a plasma and a serum of a second human being different from the first human, each of the plural red blood cells has at least one antigen thereon, each of the plasma and the serum has plural antibodies therein, the plural red blood cells agglutinate into the agglutination portion caused by an agglutination reaction, and the agglutination reaction is caused by a binding of the antigen specific to the plural antibodies.

Embodiment 18 is a crossmatching method, comprising steps of: providing a first blood sample of a first human, wherein the first sample includes one of a plasma portion and a serum portion; providing a second blood sample of a second human being different from the first human, wherein the second sample includes a blood cell portion; mixing the first and the second blood samples to form a mixed blood sample; providing a distribution space; and causing the mixed blood sample to be distributed in the distribution space due to a capillary force.

Embodiment 19 is a method according to Embodiment 18, wherein each of the plasma portion and the serum portion includes an antibody, the blood cell portion includes plural red blood cells having at least one antigen thereon, the antibody and the antigen are specifically bound in the step of mixing the first and the second blood samples so as to cause an agglutination reaction, the agglutination reaction causes the red blood cells agglutinate to form an agglutination portion in the mixed blood sample, and the method further comprises a step of distributing the mixed sample in the distribution space due to the capillary force to reveal the agglutination portion.

Embodiment 20 is a method according to Embodiments 18 or 19, wherein each of the plasma portion and the serum portion includes an antibody, the blood cell portion includes plural red blood cells having at least one antigen thereon, the antibody and the antigen are free from being bound to each other.

While this disclosure has been described in terms of what is presently considered to be the most practical and preferred embodiments, it is to be understood that the disclosure is not limited to the disclosed embodiments. Therefore, it is intended to cover various modifications and similar arrangements included within the spirit and scope of the appended claims, which are to be accorded with the broadest interpretation so as to encompass all such modifications and similar structures.

What is claimed is:

1. A test chip, comprising:
a flow channel having an inlet, an inlet surface and a channel surface, wherein at least one of the inlet surface and the channel surface has an antibody disposed thereon, the inlet receives a blood sample having an antigen, and the blood sample flows in the channel via a capillary force so that the antibody binds to the antigen and flows along with the blood sample.

2. The test chip as claimed in claim 1, wherein the blood sample enters the flow channel from the inlet, and the antibody is mixed with the blood sample and specifically bound with the antigen.

3. The test chip as claimed in claim 1, wherein the flow channel further has a far end opposite to the inlet, and the antibody bound with the antigen flows along with the blood sample to the far end.

4. The test chip as claimed in claim 1 further comprising a substrate and an upper cover, and the channel is configured between the substrate and the upper cover.

5. The test chip as claimed in claim 4, wherein the substrate has an upper surface, the upper cover has a lower surface, the upper surface and the lower surface are continuous planes, and the channel is configured between the upper surface and the lower surface.

6. The test chip as claimed in claim 5, wherein the flow channel has a channel height being a distance between the upper surface and the lower surface and less than 1 mm.

7. The test chip as claimed in claim 4, wherein the substrate has an upper surface, the upper cover has a lower surface, and the inlet surface and the channel surface are located on the upper surface.

8. The test chip as claimed in claim 4, wherein the substrate has an upper surface, the upper cover has a lower surface, the inlet surface is located on the upper surface, and the channel surface is located on the lower surface.

9. The test chip as claimed in claim 1, wherein the blood sample has a red blood cell having the antigen thereon, and the antibody is bound with the antigen so as to form an agglutination.

10. A method for identifying a blood type, comprising steps of:
providing a first and a second blood samples both obtained from a human and respectively having a first and a second red blood cells;
providing a first and a second distribution spaces;
providing an anti-A antibody and an anti-B antibody;
mixing the first and the second blood samples with the anti-A antibody and the anti-B antibody respectively;
causing the first blood sample mixed with the anti-A antibody to be distributed in the first distribution space due to a first capillary force; and
causing the second blood sample mixed with the anti-B antibody to be distribute in the second distribution space due to a second capillary force.

11. The method as claimed in claim 10, wherein the first and the second distribution spaces are separately disposed without being connected to each other.

12. The method as claimed in claim 10 further comprising steps of:
providing a third blood sample obtained from the human and having a third red blood cell;
providing a third distribution space independently disposed without being connected to either the first distribution space or the second distribution space;
providing an anti-D antibody;
mixing the third blood sample with the anti-D antibody; and
causing the third blood sample mixed with the anti-D antibody to be distributed in the third distribution space due to a third capillary force.

13. A method for identifying a blood type, comprising steps of:
providing a first and a second blood samples both obtained from a human and respectively having a first and a second serum antibodies;
providing a first and a second distribution spaces;
providing an A antigen and a B antigen;
mixing the first and the second blood samples with the A antigen and the B antigen respectively;
causing the first blood sample mixed with the A antigen to be distributed in the first distribution space due to a first capillary force; and
causing the second blood sample mixed with the B antigen to be distributed in the second distribution space due to a second capillary force.

14. The method as claimed in claim 13, wherein the first and the second distribution spaces are separately disposed without being connected to each other.

15. The method as claimed in claim 13, wherein each of the A and the B antigens is disposed on a surface of a red blood cell.

16. A method for detecting an agglutination portion in a liquid, comprising steps of:
providing a distribution space;
providing the liquid; and
causing the liquid to be distributed in the distribution space using a capillary force to reveal the agglutination portion.

17. The method as claimed in claim 16, wherein the liquid is a mixture mixing therein plural red blood cells of a first human and one of a plasma and a serum of a second human being different from the first human, each of the plural red blood cells has at least one antigen thereon, each of the plasma and the serum has plural antibodies therein, the plural red blood cells agglutinate into the agglutination portion caused by an agglutination reaction, and the agglutination reaction is caused by a binding of the antigen specific to the plural antibodies.

18. A crossmatching method, comprising steps of:
providing a first blood sample of a first human, wherein the first sample includes one of a plasma portion and a serum portion;
providing a second blood sample of a second human being different from the first human, wherein the second sample includes a blood cell portion;
mixing the first and the second blood samples to form a mixed blood sample;
providing a distribution space; and
causing the mixed blood sample to be distributed in the distribution space due to a capillary force.

19. The method as claimed in claim 18, wherein each of the plasma portion and the serum portion includes an antibody, the blood cell portion includes plural red blood cells having at least one antigen thereon, the antibody and the antigen are specifically bound in the step of mixing the first and the second blood samples so as to cause an agglutination reaction, the agglutination reaction causes the red blood cells agglutinate to form an agglutination portion in the mixed blood sample, and the method further comprises a step of:
distributing the mixed sample in the distribution space due to the capillary force to reveal the agglutination portion.

20. The method as claimed in claim 18, wherein each of the plasma portion and the serum portion includes an antibody, the blood cell portion includes plural red blood cells having at least one antigen thereon, the antibody and the antigen are free from being bound to each other.
